(19) United States
(12) Patent Application Publication
(10) Pub. No.: US 2022/0305053 A1
(43) Pub. Date: Sep. 29, 2022

(54) SKIN CONTACT TYPE MEDICAL SEMICONDUCTOR USING DIVALENT METAL OXIDE FILM FOR MUSCULOSKELETAL PAIN RELIEF AND METHOD OF DELIVERING IONS USING THE SAME

(71) Applicant: Semiconductor Lobac Co., Ltd., Seoul (KR)

(72) Inventor: SOOYUN OH, FORT LEE, NJ (US)

(21) Appl. No.: 17/388,790

(22) Filed: Jul. 29, 2021

(30) Foreign Application Priority Data
Mar. 24, 2021 (KR) 10-2021-0037683
Jun. 1, 2021 (KR) 10-2021-0071055

(51) Int. Cl. A61K 33/08; A61K 9/00; A61K 33/06; A61K 33/32; A61K 33/30; A61K 33/00; A61K 33/26; A61K 33/34

(52) U.S. Cl. CPC A61K 33/08; A61K 9/0014; A61K 33/06; A61K 33/32; A61K 33/30; A61K 33/00; A61K 33/26; A61K 33/34

(57) ABSTRACT

A medical semiconductor is used to improve musculoskeletal pain by bringing a divalent metal (magnesium alloy) oxide film semiconductor into contact with the skin.

### CROSS-REFERENCE TO RELATED APPLICATIONS

This application claims the benefit of Korean Application No. 10-2021-0037683 filed on Mar. 24, 2021 and No. 10-2021-0071055 filed on Jun. 1, 2021 with the Korean Intellectual Property Office, the disclosures of which are incorporated herein by reference in their entireties.

### BACKGROUND

Skeletal muscle pain is mainly a nerve problem. When the nerve is stimulated, an action potential is generated, depolarization occurs, and a potential difference occurs. A reflux of Na+/K+ ion channel occurs, and acetylcholine, a nociceptive substance, is secreted.

In order to solve this problem, various kinds of methods have been proposed. For example, these methods include frequency treatment for rectifying pulsation in the pain area, magnetic treatment for polarizing the depolarization of the pain area, electric potential treatment to control the potential difference by charging the lost negative (−) electricity in the pain area, and thermos treatment to warm the cold pain area due to the reverse flow of ions from sweat and potassium. However, pain relief in the painful area has been insufficient with these individual methods, and there are no treatment devices for the decomposition of nociceptive substances.

Accordingly, the present disclosure provides a method for transdermal delivery of a cofactor for pain relief without coenzyme. It also aims to simultaneously operate rectification (low frequency treatment), polarization (magnet treatment), heat (thermal treatment), charging (potential treatment), and decomposition actions.

In particular, the decomposition action is intended to provide the action of decomposing acetylcholine, a nociceptive substance, to the synapse of the pain site. Specifically, when acetylcholine is hydrolyzed, it is decomposed into choline and acetic acid to relieve pain. In order to activate this decomposition, a supply of divalent metal ions is required. The present disclosure relates to a semiconductor containing magnesium alloy, which includes 90% or more of magnesium and zinc, iron, copper, manganese, silicon, aluminum, etc. as a main material for directly delivering divalent metal ions in a transdermal delivery method and medical devices or health-promoting devices including the same. Through this delivery method, the above-mentioned five actions can be delivered at the same time to quickly relieve and eliminate pain.

Most skeletal muscle pain is a nerve problem, and this pain is induced when acetylcholine, a nociceptive substance, is secreted at the synapse of nerve cells in the pain area. Pain can be relieved or eliminated when this acetylcholine is decomposed into choline and acetic acid as shown in the following reaction formula.

Acetylcholine+H2O=choline+acetic acids [Reaction Formula]

Acetylcholine esterase enzyme, coenzyme, and cofactor are required to degrade acetylcholine through this reaction. In particular, coenzymes and cofactors have been recognized as essential for degradative activation, and conventionally, they have been provided by oral administration. However, the oral administration method requires both a coenzyme and a cofactor, and thus the oral administration method is often cumbersome. In addition, it is necessary to study a method that can provide the aforementioned five actions (for example, rectification, polarization, warming, charging and decomposition).

### RELATED ART DOCUMENT

### Patent Document

Korean Patent Publication No. 10-0389703

### SUMMARY

The present inventor developed a medical semiconductor that is used to reduce musculoskeletal pain by contacting a divalent metal (magnesium alloy) oxide film semiconductor with the skin.

Specifically, the present disclosure intends to provide pain relief and improvement only by delivery of cofactors through transdermal delivery without coenzymes, as will be described later. In particular, it is intended to provide a semiconductor (MOS) by forming an oxide film on the metal surface that is manufactured by anodizing a magnesium alloy without sealing to deliver ions through the transdermal layer.

The metal oxide semiconductor of the present disclosure has the same structure as the half electrolytic capacitor. The structure of a general electrolytic capacitor is composed of metal+insulator+electrolyte, while the half electrolytic capacitor is composed of metal+insulator without electrolyte. When such a half-capacitor is in contact with the skin, body fluid acts as an electrolyte, and a structure of metal+insulator+body fluid electrolyte is formed, so that it can function as a complete electrolytic capacitor.

Such a semiconductor is made of an alloy such as 90% magnesium or more and silicon. When the alloy bundle of magnesium and silicon is oxidized, a silicon dioxide film is formed on the metal surface, which becomes a metal oxide film semiconductor.

During anodization, the alloy is immersed in an electrolytic cell containing an alkali solution produced through anodizing and cathodic reduction, and a fine electric charge is transferred by contact with the electrolyte of body fluids such as sweat on the skin. The electrolyte in the electrolyzer and the electrolyte in the skin body fluid are the same alkaline liquid.

When these semiconductors are in close contact with the skin of the painful area they act as electrolytic capacitors for rectification/polarization/charging/heating/decomposition, providing effects such as low-frequency treatment/magnet treatment/potential treatment/thermal treatment/decomposition treatment at the same time. Specifically, when such a semiconductor is in close contact with the skin of a pain site, magnesium can be delivered directly through the skin without oral administration. Moving from a high concentration to a low concentration, the ions are transported through the electrochemical gradient of the inside and outside of the skin.

An embodiment of the present disclosure provides a transdermal delivery method of transporting metal ions from the magnesium-based alloy article into a skin by bringing the magnesium-based alloy article into contact with the skin of the human body, the magnesium-based alloy article comprising magnesium-based alloy member and an oxide film formed on a portion of the surface of the magnesium-based alloy member.

As an example, the magnesium-based alloy member may include manganese.

As an example, the magnesium-based alloy member may include at least one selected from the group consisting of aluminum, zinc, manganese, silicon, iron, and copper.

As an example, the magnesium-based alloy member may include 0.1 to 0.3% by weight of aluminum, 0.2 to 0.4% by weight of zinc, 1.3 to 2.5% by weight of manganese, 0.01 to 0.2% by weight of silicon, 0.01 to 0.1% by weight of iron, 0.01 to 0.1% by weight of copper, and the remainder of magnesium.

As an example, the oxide may comprise at least one selected from the group consisting of magnesium oxide, silicon oxide, and aluminum oxide.

As an example, the magnesium-based alloy member may come into contact with the skin of the human body so that the body fluid acts as an electrolyte to allow the magnesium-based alloy member to operate as a semiconductor comprising a magnesium-based alloy member, an oxide film, and an electrolyte.

As an example, the magnesium-based alloy member may be applied to any one of a patch, an accessory, a waistband, a net, bedding, a pad, a metal mask, an insole, a mat, or a warmer.

According to one embodiment of the present disclosure, delivery of only cofactors, even without coenzyme for pain relief, can provide an excellent pain relief effect.

According to one embodiment of the present disclosure, it is possible to provide a semiconductor capable of using a bodily fluid electrolyte when in contact with the skin of a human body without an electrolyte.

According to an embodiment of the present disclosure, it is possible to provide a semiconductor capable of delivering ions into the body of the contact site through the skin without oral administration (Targeting Transdermal Ion delivery system).

### BRIEF DESCRIPTION OF THE DRAWINGS

FIG. 1 shows a magnesium-based alloy article in the form of a patch according to an embodiment of the present disclosure.

FIG. 2 shows a magnesium-based alloy article in the form of a waist belt according to an embodiment of the present disclosure.

FIG. 3 shows a skin-contacting accessory type magnesium-based alloy article according to an embodiment of the present disclosure.

FIG. 4 is a schematic diagram depicting a method of applying the magnesium-based alloy article to back pain, hip pain, and sciatic pain according to an embodiment of the present disclosure.

FIG. 5 is a schematic diagram depicting a method of applying a magnesium-based alloy article to shoulder pain according to an embodiment of the present disclosure.

FIG. 6 is a schematic diagram depicting a method of applying a magnesium-based alloy article to forearm pain according to an embodiment of the present disclosure.

FIG. 7 is a schematic diagram depicting a method of applying a magnesium-based alloy article to plantar heel pain according to an embodiment of the present disclosure.

FIG. 8 is a schematic diagram depicting a method of applying a magnesium-based alloy article to wrist pain according to an embodiment of the present disclosure.

FIG. 9 is a schematic diagram depicting a method of applying a magnesium-based alloy article to tailbone pain according to an embodiment of the present disclosure.

FIG. 10 is a schematic diagram depicting a method of applying a magnesium-based alloy article to knee pain according to an embodiment of the present disclosure.

FIG. 11 is a schematic diagram depicting a method of applying a magnesium-based alloy article to popliteal pain according to an embodiment of the present disclosure.

FIG. 12 is a schematic diagram depicting a method of applying a magnesium-based alloy article to back pain according to an embodiment of the present disclosure.

FIG. 13 is a view illustrating a state in which the magnesium-based alloy article according to an embodiment of the present disclosure is applied to a patch.

FIG. 14 is a view illustrating a state in which the magnesium-based alloy article according to an embodiment of the present disclosure is applied to the shoe insole.

FIG. 15 is a view illustrating a state in which the magnesium-based alloy article according to an embodiment of the present disclosure is applied to the floor plate.

FIG. 16 is a cross-sectional view of a warmer using a magnesium alloy member according to an embodiment of the present disclosure.

### DETAILED DESCRIPTION

The terms used in the present disclosure are only used to describe specific embodiments, and they are not intended to limit the present disclosure. The singular expression includes the plural expression unless the context clearly dictates otherwise. In the present disclosure, terms such as “include” or “have” are intended to designate that the features, components, etc. described in the specification are present, but not to mean that one or more other features or components may not be present or may be not added.

Unless defined otherwise, all terms used herein, including technical or scientific terms, have the same meaning as commonly understood by one of ordinary skill in the art to which this invention belongs. Terms such as those defined in commonly used dictionaries should be interpreted as having a meaning consistent with the meaning in the context of the related art and should not be interpreted in an ideal or excessively formal meaning unless explicitly defined in the present disclosure.

Skeletal muscle pain is mainly a neurological problem, and when any stimulus is applied to the skeletal muscle, action potentials are generated, and potential difference occurs during depolarization. Then, reflux of Na+/K+ ion channels occurs, and nociceptive substances are secreted.

Several devices have been proposed to treat such pain. In particular, a frequency treatment device has been proposed to rectify the pulsation caused by the action potential in the pain area. In addition, a magnetic treatment device for polarizing the depolarization of the pain region has been proposed, and an electric potential treatment device for controlling the potential difference by charging the lost negative (−) electricity in the pain region has been proposed. In addition, a thermos treatment device has been proposed to prevent body temperature from falling due to the backflow of ions (Na+/K+) from which sweat enters and potassium comes out from the nociceptive site, but a device that performs these actions at the same time has not been proposed. In particular, there was nothing related to the decomposition treatment of nociceptive substances secreted on the synapse.

Unlike the conventional Metal Oxide Semiconductor structure having a metal, an insulator, and an electrolyte (metal+insulator+electrolyte), an embodiment of the present disclosure may have a structure of a metal and an insulator (metal+insulator) without an electrolyte.

When such a device comes into contact with the skin, the body fluid becomes an electrolyte solution, and the structure of the electrolytic capacitor is complete between the metal, the insulator, and the body fluid electrolyte (metal+insulator+body fluid electrolyte). When the semiconductor is in close contact with the body fluid of the skin at the pain site, it becomes a body fluid electrolyte with base, and thus ions can be delivered by the difference in concentration.

Medical Semiconductors (Magnesium-Based Alloy Articles)

One embodiment of the present disclosure provides a magnesium-based alloy article. The magnesium-based alloy article of the present disclosure may be manufactured as a metal oxide film semiconductor by anodizing the magnesium alloy.

In an example, the magnesium-based alloy article may comprise magnesium-based alloy member and an oxide film formed on a portion of the surface of the magnesium-based alloy member.

In this case, the magnesium-based alloy member may include manganese. More specifically, the magnesium-based alloy member may further include at least one selected from the group consisting of aluminum, zinc, manganese, silicon, iron, and copper.

Preferably, the magnesium-based alloy member may include 0.1 to 0.3% by weight of aluminum, 0.2 to 0.4% by weight of zinc, 1.3 to 2.5% by weight of manganese, 0.01 to 0.2% by weight of silicon, 0.01 to 0.1% by weight of iron, 0.01 to 0.1% by weight of copper, and the remainder of magnesium.

Most preferably, the magnesium-based alloy member may include 0.2% by weight of aluminum, 0.3% by weight of zinc, 1.3 to 2.5% by weight of manganese, 0.1% by weight of silicon, 0.05% by weight of iron, 0.05% by weight of copper, and the remainder of magnesium.

As an example, the oxide may comprise at least one selected from the group consisting of magnesium oxide, silicon oxide, and aluminum oxide. As described above, an additional metal oxide may be formed because of the metal contained in addition to magnesium. The content of the corresponding metal oxide having a small content is also small, and thus a significant effect may not be provided.

As an example, the magnesium-based alloy member may come into contact with the skin of the human body so that the body fluid acts as an electrolyte. Thus, the magnesium-based alloy member may be a semiconductor comprising a magnesium-based alloy member, an oxide film, and an electrolyte.

In particular, the main metal of the alloy article may include 90% or more by weight of magnesium.

When the magnesium-based alloy article of the present disclosure manufactured in this scheme is in close contact with the skin of the painful part of the human body, it becomes a structure of an intact half electrolysis condenser. Thus, it performs various actions of a medical semiconductor (for example, rectification/polarization/charging/heating/decomposition) to provide a variety of treatments (for example, low-frequency treatment/magnet therapy/potential therapy/thermal therapy/decomposition therapy).

An article or device according to an embodiment of the present disclosure is a device for coping with the secretion of acetylcholine. A magnesium alloy (Mg, Mn, Zn, Si, Al, Fe, Cu, etc.) may be anodized to prepare a bivalent metal oxide semiconductor (MOS). The device is in direct contact with the painful area to deliver cofactors (magnesium, zinc, iron, copper, manganese, etc.) that rapidly activate the decomposition of pain-inducing substances (acetylcholine).

The medical semiconductor according to an embodiment of the present disclosure is manufactured by anodizing alloy such as manganese (Mn), zinc (Zn), silicon (Si), aluminum (Al), iron (Fe), and copper (Cu) magnesium (Mg) in addition to magnesium (Mg) to form a metal oxide semiconductor (MOS).

A described above, when the magnesium alloy (metal) is anodized, a metal oxide such as a magnesium oxide (MgO) film, a silicon dioxide (SiO₂) film, or an alumina oxide (Al₂O₃) film formed on the surface of the metal becomes an insulating member or insulator, and thus the device may become a metal oxide semiconductor (MOS) in the form of a half conductor and a half non-conductor that becomes a conductor only when the electrolyte is in contact.

The present disclosure provides a method for directly delivering magnesium, zinc, iron, copper, manganese, etc. (targeting transdermal ion delivery system), which are cofactors of acetyl choline esterase enzyme, through the skin without oral administration. In order to perform this method, the semiconductor for medical device, which is an embodiment of the present disclosure, is brought into close contact with or in contact with the skin of the painful area to cause a chemical reaction with body fluid (movement from a high concentration to a low concentration of the ions by the electrochemical gradient inside and outside the skin), thereby allowing the delivery of ions.

As an example, in the process of anodic oxidation of magnesium alloys, magnesium alloys (including Zn, Fe, Cu, Mn, Si, Al, etc.) are immersed in an electrolytic bath containing alkaline solutions including Na⁺, K⁺ etc. so that the ions in the alloy and the ions in the electrolytic bath are replaced, and thus an oxide film is formed on the surface of the magnesium alloy to form a semiconductor. Assuming that this semiconductor is taken out of the electrolytic bath during electrolysis and brought into contact with body fluid electrolytes (Na⁺ and K⁺), electrolysis occurs again through contact with body fluids electrolytes (Na, K, Cl, Ca, Mg, P, Bicarbonate, Protein, etc.). Therefore, electric charge is transferred, and eventually delivery of microscopic ions can be achieved. Here, the electrolyte of electrolytic bath and the body fluid are the same alkaline liquid, which is Na⁺ and K⁺.

As such, magnesium, zinc, iron, copper, manganese, etc. made of divalent metal oxide semiconductors are substances that activate the hydrolysis of acetylcholine esterase enzyme, which is a nociceptive substance, and are used as coenzymes as well as cofactors.

In the present disclosure, a magnesium-based alloy is anodized to make a semiconductor. Specifically, when magnesium and a manganese-based alloy (metal) are oxidized, a magnesium oxide (MgO) film, a silicon dioxide (SiO₂) film, an alumina oxide (Al₂O₃) film, etc. are formed on the metal surface to form a metal oxide film semiconductor (MOS).

Transdermal Delivery of Cofactors

The metal oxide film semiconductor formed in this scheme is brought into contact with the skin of a skeletal muscle pain site of the human body to deliver cofactor for accelerating (activating) the hydrolysis of acetylcholine esterase enzyme through the skin, thereby rapidly alleviating pain.

Magnesium is used for the purpose of supplying the electrochemical function of increasing the human body current and the function of activating enzymes. In addition, manganese and iron may act as oxidizing agents that rapidly promote the generation of ions in the human body. In addition, copper and zinc may function as redox enzymes in the body in low oxidation states.

Targeting transdermal ion delivery system is a method of directly delivering a divalent metal element such as magnesium (cofactor) through the skin without oral delivery. The medical semiconductor of the present disclosure is brought in contact with or in close contact with the skin of a pain area to induce chemical reaction with the body fluid, thereby performing the ion delivery.

Decomposition of Acetylcholine

The nociceptive substance acetylcholine is released at the synapse of the pain site. If it is decomposed, the pain-inducing action is lost. The minerals are needed as cofactors to rapidly activate the decomposition.

Acetylcholine+H₂O=choline+acetic acids [Reaction formula]

When acetylcholine, which is a pain-inducing substance in the above reaction formula, is hydrolyzed, it is decomposed into choline and acetic acid to improve pain. The decomposition can be rapidly activated through the supply of divalent metal ions (cofactor). However, in a prior art, oral administration, which could not be targeted for treatment, was not effective.

The metal oxide film semiconductor is made by oxidizing a magnesium alloy (Mg, Mn, Zn, Si, Al, Fe, Cu, etc.).

In an embodiment of the present disclosure, the main metal of the semiconductor may be a magnesium alloy, which includes 90% or more of magnesium. The medical semiconductor of the present disclosure, in which the above-described ions can be directly targeted and delivered to the skin at the contact site through the skin, can be provided as various articles.

Hereinafter, the present disclosure is described in detail.

The semiconductor article of the present disclosure can be used in a patch-type, a skin contact based-accessory type, or a waist belt-type.

As an example, it may be in the form of a patch, and the patch member can be attached for 50 minutes to about 8 hours to improve pain, and as another example, necklaces or bracelets can be contacted for short moments to improve pain while the patch is used for long time.

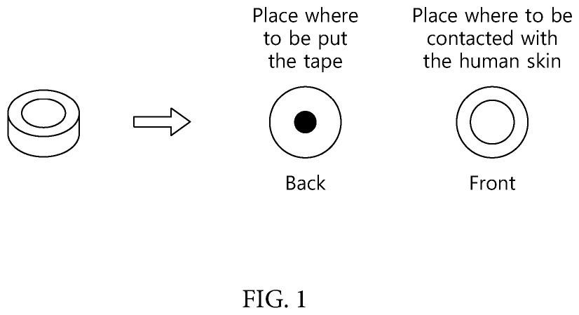

FIG. 1 shows a magnesium-based alloy article in the form of a patch according to an embodiment of the present disclosure.

As one embodiment, the patch is manufactured in a rectangular shape or a circular shape, and the size can be adjusted to about 10 D mm, 20 D mm, 30 D mm, etc. in diameter, and it can also be manufactured in the in the form of a wire, which can be manufactured in various shapes.

The thickness may be about 1 to 3 mm, but is not limited thereto.

The patch site may be a pain site, for example musculature pain site, such as a spinal afferent nerve site, a back, shoulder, knee, sacrum, hip joint, sciatic bone, calf, popliteal muscle, or the sole of the foot.

As for the contact method, the semiconductor patch is attached to the non-woven tape or cotton tape, and if there is body hair, after shaving the same, they are attached to the skin at the painful area directly or with bandage.

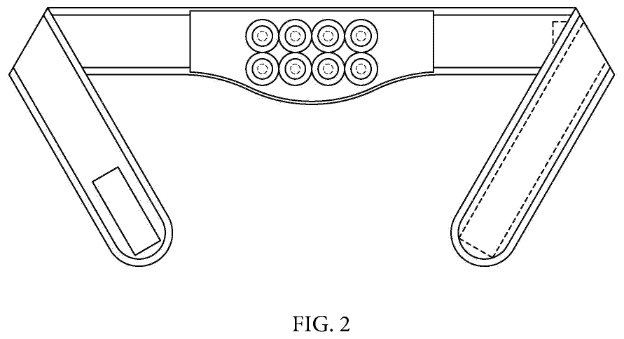

Furthermore, a piece of a bivalent metal oxide semiconductor is attached to the Velcro band to which the heat source of the rechargeable battery is applied. Such a device may contact the back pain area. FIG. 2 shows a magnesium-based alloy article in the form of a waist belt according to an embodiment of the present disclosure.

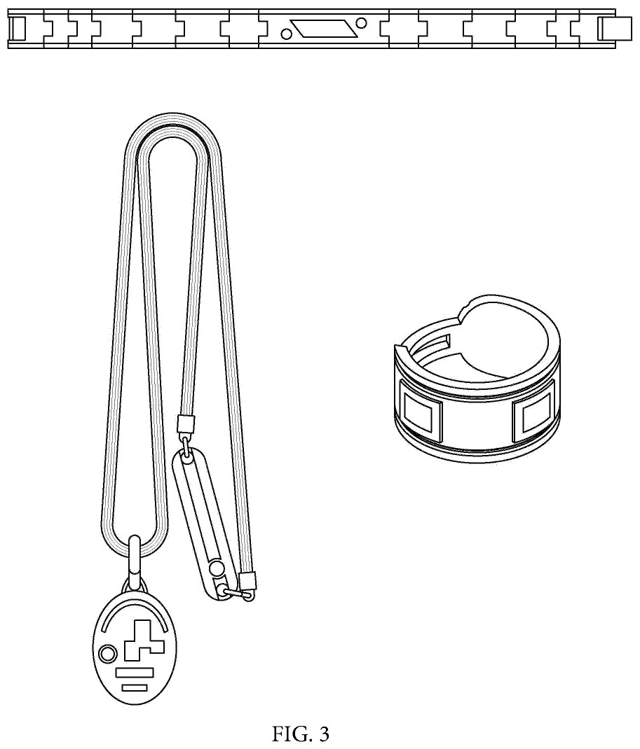

In addition, skin-contact type accessories may have various uses and effects in addition to the musculoskeletal pain improvement effect. In particular, wearing a necklace type pendant can control heart rate and eliminate respiratory disorders, and it can also encourage mental stability by awakening from brain waves. In addition, rings and bracelets stimulate peripheral nerves to promote blood flow, thereby improving toothache due to periodontitis within about 30 minutes due to its anti-inflammatory action. Inflammatory skin such as bedsores can be improved within 24 hours by dehydrogenation. A pendant is applied in the form of a necklace so that a piece of bivalent metal oxide semiconductor covers the area of pain in chest pain. FIG. 3 shows a skin-contacting accessory type magnesium-based alloy article according to an embodiment of the present disclosure.

The divalent metal oxide semiconductor of the present disclosure, which enables metal ions to be delivered directly into the body at the skin contact site through the skin, is a non-toxic, biocompatible essential material. Alkaline solution Na+, K+ in an electrolytic bath such as body fluid is used in the production process. It allows targeting only the painful area so that stability is maintained, and it can be implemented at an industrially low price.

In addition, in order to provide chemotherapy other than the above-mentioned four medical devices (low frequency therapy device, magnetic therapy device, potential therapy device, and heat therapy device) and 90% or more aluminum alloy patch, trace amounts of zinc, iron, copper manganese, silicon, aluminum, etc. are added to more than 90% magnesium to form a divalent metal oxide film semiconductor, thereby providing a more effective treatment device.

When the magnesium/manganese alloy is anodized, it becomes a metal oxide semiconductor. When this semiconductor is attached to or brought into contact with body fluid of the skin at the pain site, microscopic charge transfer occurs within the body fluid electrolyte. Charge transfer of magnesium, zinc, iron, copper, manganese, etc. works as a cofactor that activates the decomposition of acetylcholine, a nociceptive substance at the synapse of the pain site, thereby improving pain quickly and easily. In the prior art, the conventional device uses 90% or more of aluminum to focus on dehydrogenation of the painful area, but the present disclosure increased the magnesium content to 90% or more to activate as a cofactor.

Hereinafter, a pain site to which the present disclosure can be applied and an application method is described.

### Example 1: Improvement of Back Pain

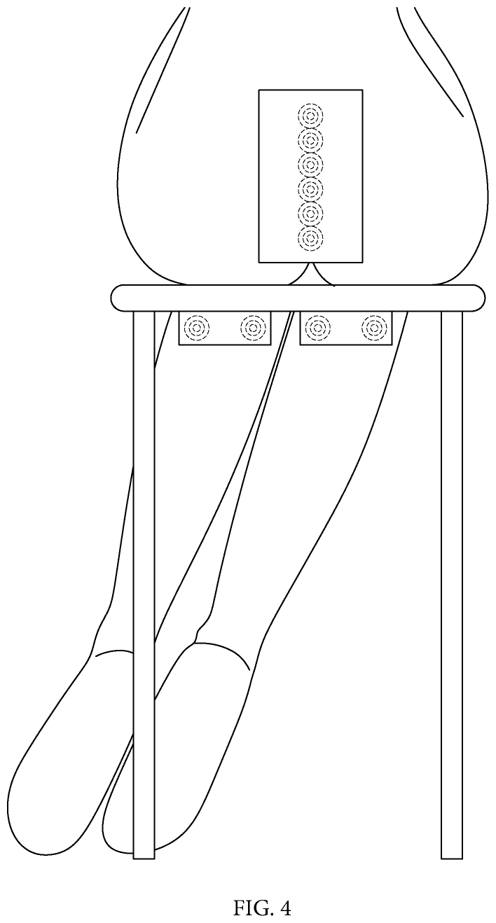

Pain can be improved by attaching the above-mentioned semiconductor patch to the upper part of the coccyx for back pain, lumbar stenosis pain, hip joint pain, sciatic pain, weakness of the lower extremities, etc. FIG. 4 is a schematic diagram depicting a method of applying the magnesium-based alloy article to back pain, hip pain, and sciatic pain according to an embodiment of the present disclosure. As shown in FIG. 4, the patches are attached in a row so that pain can be improved by attaching the patches to the upper part of the coccyx for back pain, lumbar stenosis, hip joint pain, sciatic pain, and weakness in the lower extremities.

### Example 2: Shoulder Pain

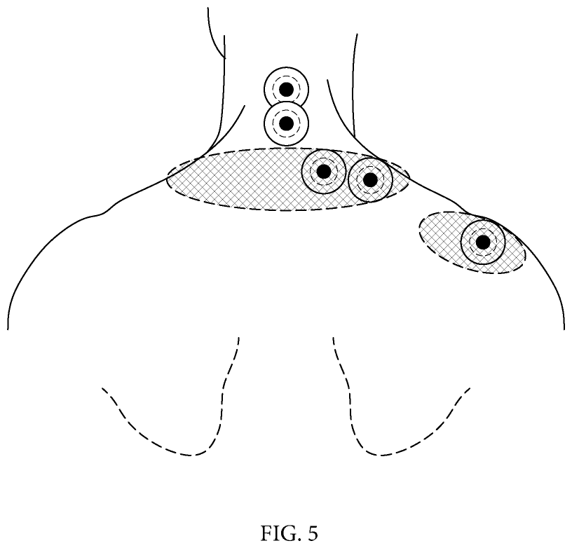

In case of shoulder pain, attaching the above-mentioned semiconductor patch to the shoulder can improve the pain. FIG. 5 is a schematic diagram depicting a method of applying a magnesium-based alloy article to shoulder pain according to an embodiment of the present disclosure. As shown in FIG. 5, attaching a patch to a place where wrinkles are formed when the head is tilted back, a dent at the tip of the shoulder, and the like can improve the pain.

### Example 3: Forearm Pain

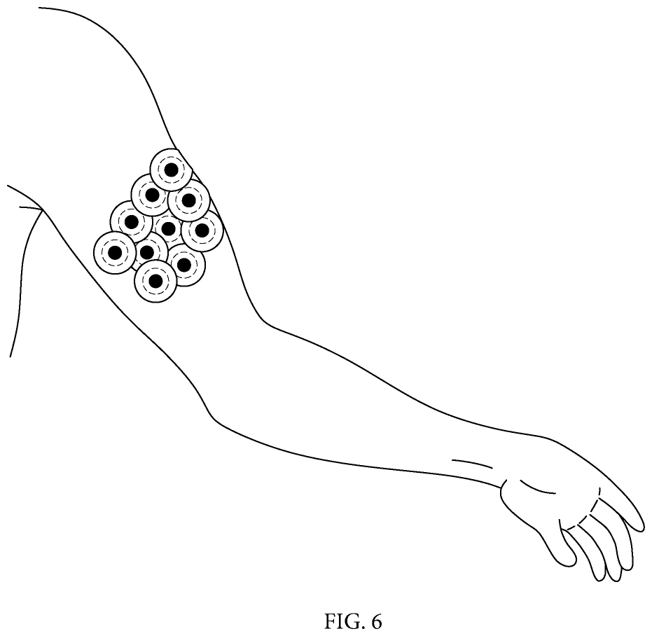

In case of forearm pain, attaching the above-mentioned semiconductor patch to the forearm can improve the pain. FIG. 6 is a schematic diagram depicting a method of applying a magnesium-based alloy article to forearm pain according to an embodiment of the present disclosure. As shown in FIG. 6, attaching 2 to 3 patches to the end of the wrinkled area when the arm is bent at intervals of 3 days can improve the pain.

### Example 4: Plantar Heel Pain

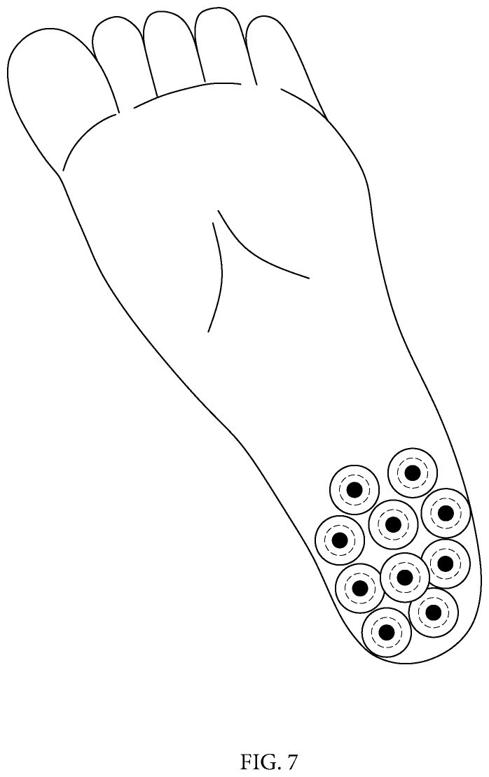

In case of plantar heel pain, attaching the above-mentioned semiconductor patch to the plantar heel can improve the pain. FIG. 7 is a schematic diagram depicting a method of applying a magnesium-based alloy article to plantar heel pain according to an embodiment of the present disclosure. As shown in FIG. 7, attaching the patch to about 10 pain points on the plantar heel can improve the pain. For example, on both feet the patch is transferred in the morning and evening, or every 8 hours, and 6 patches are attached above the tailbone, because the 4th and 5th sacrum vertebrae are the problem.

### Example 5: Wrist Pain

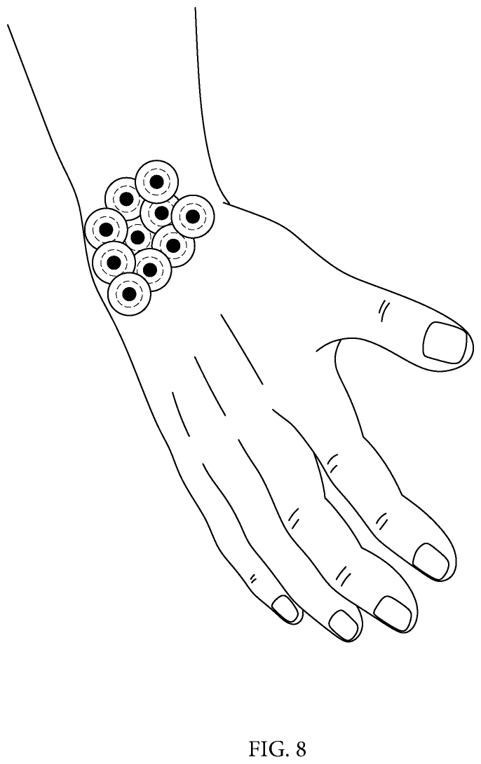

In case of wrist pain, attaching the above-mentioned semiconductor patch to the wrist can improve the pain. FIG. 8 is a schematic diagram depicting a method of applying a magnesium-based alloy article to wrist pain according to an embodiment of the present disclosure. As shown in FIG. 8, attaching a plurality of patches to the painful area of the wrist can improve the pain.

### Example 6: Tailbone Pain

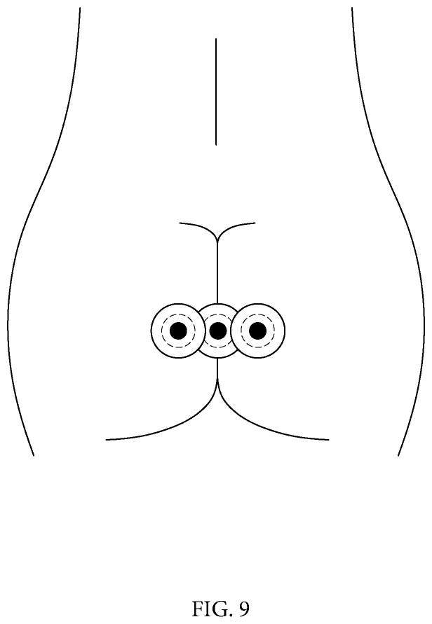

In case of tailbone pain, attaching the above-mentioned semiconductor patch to the tailbone can improve the pain. FIG. 9 is a schematic diagram depicting a method of applying a magnesium-based alloy article to tailbone pain according to an embodiment of the present disclosure. As shown in FIG. 9, attaching 2 to 3 patches to the painful area of the tailbone for 8 hours can improve the pain.

### Example 7: Knee Pain

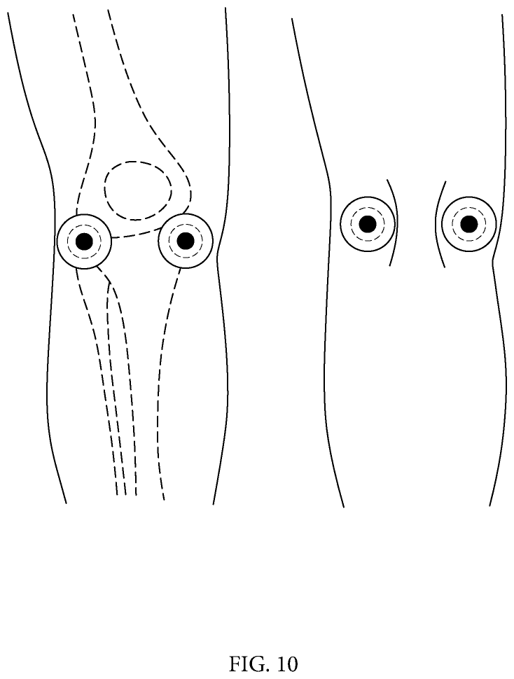

In case of knee pain, attaching the above-mentioned semiconductor patch to the knee can improve the pain. FIG. 10 is a schematic diagram depicting a method of applying a magnesium-based alloy article to knee pain according to an embodiment of the present disclosure. As shown in FIG. 10, attaching a patch to the knee pain area can improve the pain.

### Example 8: Popliteal Pain

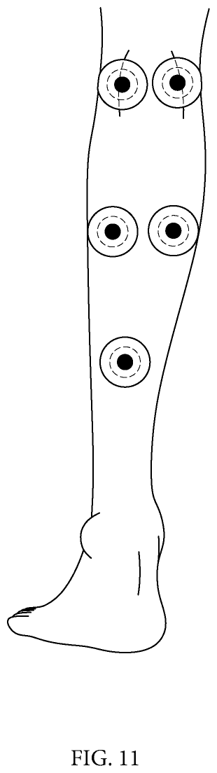

In case of popliteal pain, attaching the above-described semiconductor patch to the popliteal area can improve the pain. FIG. 11 is a schematic diagram depicting a method of applying a magnesium-based alloy article to popliteal pain according to an embodiment of the present disclosure. As shown in FIG. 11, attaching the patch to the wrinkled area behind the knee, the bulging area of the shin, and the area marked with ‘A’ can improve the pain.

### Example 9: Musculoskeletal Pain

In case of musculoskeletal pain, finding the pain point and attaching the above-mentioned semiconductor patch to the pain point can improve the pain.

### Example 10: Back Pain

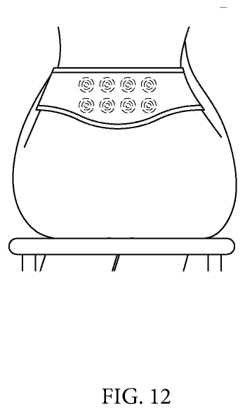

Contacting a device in which the above-mentioned semiconductor article to the Velcro band including the heat source of the rechargeable battery to the back pain area can improve the pain. FIG. 12 is a schematic diagram depicting a method of applying a magnesium-based alloy article to back pain according to an embodiment of the present disclosure. As shown in FIG. 12, wearing a magnesium alloy semiconductor band on the waist region can improve the pain.

### Example 11: Finger Pain

In case of finger pain, wearing a ring-shaped accessory made of the above-described semiconductor for finger pain can improve the pain.

### Example 12: Wrist Pain

In case of wrist pain, wearing the bracelet-type accessory made of the above-described semiconductor can improve the pain.

### Example 13: Ankle Pain

In case of ankle pain, wearing the ankle-shaped accessory made of the above-described semiconductor during ankle pain can improve the pain.

### Example 14: Chest Pain

In case of chest pain, wearing the necklace-type accessory made of the above-described semiconductor can improve the pain.

### Example 15: Bedsores

Contacting the above-described net shape or patch made of the above-mentioned semiconductor to the bedsore area may improve the bedsore or its pain.

### Example 16: Bedding

Attaching the above-described semiconductor to a portion of the bedding that may be in contact with the skin may improve the pain.

### Example 17: Mat

Attaching the above-described semiconductor to a portion of the mat that may be in contact with the skin may improve the pain.

### Example 18: Mask

Wearing the metal mask made of the above-described semiconductor during facial pain may improve the pain.

### Example 19: Patch

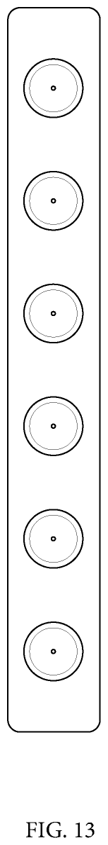

As described above, attaching the patch made of the semiconductor on the painful area when the lower back pain, hip joint pain, sciatic pain, coccyx pain, or lower extremity weakness is present can improve the pain. FIG. 13 is a view illustrating a state in which the magnesium-based alloy article according to an embodiment of the present disclosure is applied to a patch.

### Example 20: Insole

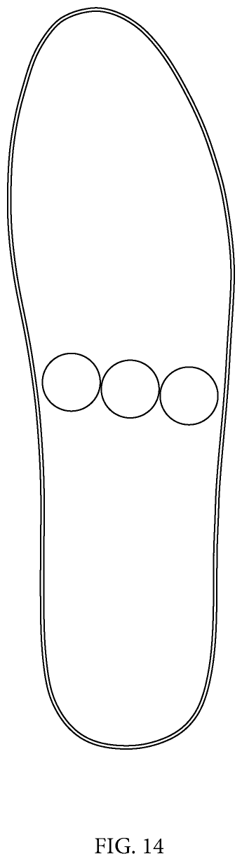

Attaching the above-described semiconductor to a portion of the insole of a shoe that may be in contact with the skin may improve the pain. FIG. 14 is a view illustrating a state in which the magnesium-based alloy article according to an embodiment of the present disclosure is applied to the shoe insole.

### Example 21: Floor Plate

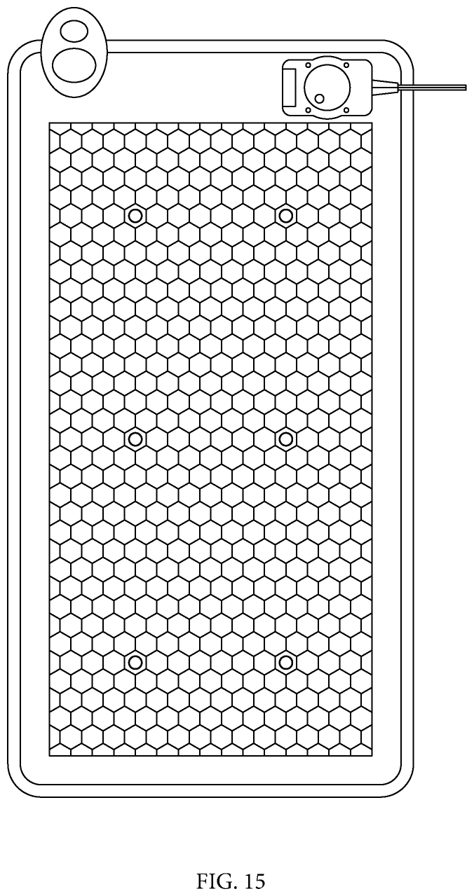

Attaching the above-described semiconductor to a portion of the floor plate that may be in contact with the skin can improve the pain. FIG. 15 is a view illustrating a state in which the magnesium-based alloy article according to an embodiment of the present disclosure is applied to the floor plate.

### Example 22: Warmer

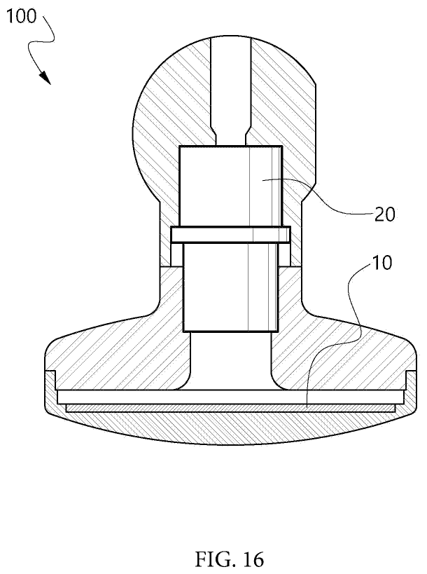

A warmer (heater) can be manufactured using the magnesium alloy member of the present disclosure. The external shape of the warmer is not particularly limited, but may include a handle part so that a user can grip and use the warmer and a heat transfer part connected from the handle part. FIG. 16 is a cross-sectional view of a warmer using a magnesium alloy member according to an embodiment of the present disclosure. As shown in FIG. 16, it includes carbon fiber heating element 10 in the heat transfer part within the warmer 100 to supple power from the outside, generating heat from carbon fiber heating element 10. The heat is transferred to the magnesium alloy member. Thus, in addition to heat, the effect of the above-described magnesium alloy member is provided to the use area of the user who operates the warmer 100. In addition, a vibration motor 20 and a control unit (not shown) capable of driving and controlling the vibration motor 20 are included inside the handle part. Power is supplied from the outside to drive the vibration motor 20 so that an additional vibration effect is provides to the used area of users. However, as long as it is a warmer capable of realizing the effect of a magnesium alloy member along with heat, it is not limited to this description, and it can be changed into various shapes and structures.

Although the above has been described with reference to the preferred embodiments of the present disclosure, it will be understood that those skilled in the art can variously modify and change the present disclosure without departing from the spirit and scope of the present disclosure as set forth in the following claims.

What is claimed is:

1. A transdermal delivery method of transporting metal ions from the magnesium-based alloy article into a skin by bringing the magnesium-based alloy article into contact with the skin of the human body, the magnesium-based alloy article comprising magnesium-based alloy member and an oxide film formed on a portion of the surface of the magnesium-based alloy member.

2. The method of claim 1, wherein the magnesium-based alloy member includes manganese.

3. The method of claim 1, wherein the magnesium-based alloy member includes at least one selected from the group consisting of aluminum, zinc, manganese, silicon, iron, and copper.

4. The method of claim 1, wherein the magnesium-based alloy member includes 0.1 to 0.3% by weight of aluminum, 0.2 to 0.4% by weight of zinc, 1.3 to 2.5% by weight of manganese, 0.01 to 0.2% by weight of silicon, 0.01 to 0.1% by weight of iron, 0.01 to 0.1% by weight of copper, and the remainder of magnesium.

5. The method of claim 1, wherein the oxide comprises at least one selected from the group consisting of magnesium oxide, silicon oxide, and aluminum oxide.

6. The method of claim 5, wherein the magnesium-based alloy member comes into contact with the skin of the human body so that the body fluid acts as an electrolyte to allow the magnesium-based alloy member to operate as a semiconductor comprising a magnesium-based alloy member, an oxide film and an electrolyte.

7. The method of claim 6, wherein the magnesium-based alloy member is applied to any one of a patch, an accessory, a waistband, a net, bedding, a pad, a metal mask, an insole, a mat, a mat, and a warmer.

8. The method of claim 4, wherein the magnesium-based alloy member comes into contact with the skin of the human body so that the body fluid acts as an electrolyte to allow the magnesium-based alloy member to operate as a semiconductor comprising a magnesium-based alloy member, an oxide film and an electrolyte.

9. The method of claim 8, wherein the magnesium-based alloy member is applied to any one of a patch, an accessory, a waistband, a net, bedding, a pad, a metal mask, an insole, a mat, a mat, and a warmer.

10. The method of claim 3, wherein the magnesium-based alloy member comes into contact with the skin of the human body so that the body fluid acts as an electrolyte to allow the magnesium-based alloy member to operate as a semiconductor comprising a magnesium-based alloy member, an oxide film and an electrolyte.

11. The method of claim 10, wherein the magnesium-based alloy member is applied to any one of a patch, an accessory, a waistband, a net, bedding, a pad, a metal mask, an insole, a mat, a mat, and a warmer.

12. The method of claim 2, wherein the magnesium-based alloy member comes into contact with the skin of the human body so that the body fluid acts as an electrolyte to allow the magnesium-based alloy member to operate as a semiconductor comprising a magnesium-based alloy member, an oxide film and an electrolyte.

13. The method of claim 12, wherein the magnesium-based alloy member is applied to any one of a patch, an accessory, a waistband, a net, bedding, a pad, a metal mask, an insole, a mat, a mat, and a warmer.

14. The method of claim 1, wherein the magnesium-based alloy member comes into contact with the skin of the human body so that the body fluid acts as an electrolyte to allow the magnesium-based alloy member to operate as a semiconductor comprising a magnesium-based alloy member, an oxide film and an electrolyte.

15. The method of claim 14, wherein the magnesium-based alloy member is applied to any one of a patch, an accessory, a waistband, a net, bedding, a pad, a metal mask, an insole, a mat, a mat, and a warmer.
